(19) United States
(12) Patent Application Publication
(10) Pub. No.: US 2025/0189489 A1
(43) Pub. Date: Jun. 12, 2025

(54) WATER QUALITY DETECTION IN STATIC WATER METER USING DEEP LEARNING

(71) Applicant: Honeywell International Inc., Charlotte, NC (US)

(72) Inventors: Debabrata Pal, Kolkata (IN); Sameer Khaladkar, Hyderabad (IN); Nishitha Kethidi, Warangal (IN); Avinav Srivastava, Bangalore (IN)

(21) Appl. No.: 18/532,384

(22) Filed: Dec. 7, 2023

(51) Int. Cl. G01N 29/024; G01N 29/44; G01N 33/18

(52) U.S. Cl. CPC G01N 29/024; G01N 29/44; G01N 33/18

(57) ABSTRACT

Methods and systems for detecting water quality, can involve classifying the quality of water using a water meter with respect to data indicative of ultrasonic time-of-flight (ToF) change behavior due to a mixed or combination of impurities in the water, and utilizing a sequential learning unit for classification of impurities in the water. The data indicative of ultrasonic time-of-flight change behavior can be obtained from one or more ultrasonic sensors associated with the water meter. The impurities in the water can be classified by the sequential learning unit as water quality parameters including one or more of, for example, TDS (Total Dissolved Solids), ph level, chlorine residual data, turbidity information, and total organic carbon values. The data can be transmitted to a user through a radio frequency frame.

### TECHNICAL FIELD

Embodiments are generally related to the field of water quality detection. Embodiments also relate to the use of ultrasonic sensors and static water meters for detecting impurities in water.

### BACKGROUND

Poor water quality is a significant issue and currently faced by most of the countries across the word, this impacts on health of country citizens and adversely affect their productivity. In many countries, water utilities who already take care of purification, faces noncompliance due to impurity getting dissolved during distribution, this happens due to man made mistakes or due to natural calamity. Water quality detection at metering level will help consumer as well as utilities to take appropriate action when impurities increases. Utilities are installing different water sensing sensors along with alarms systems to handle this problem statements but to have accurate measure of change in water quality there is a need of intelligent software solution which would be able to train itself for existing water quality and triggers alarms when different kind of dissolved impurities are detected.

Poor water quality is a significant issue that can affect many countries around the world. This problem has a direct impact on the health of citizens and hampers their productivity. In several countries, even though water utilities are responsible for water purification, they often struggle with noncompliance issues as impurities can enter the water during distribution, either due to human errors or natural disasters.

To address this challenge, the implementation of water quality detection at the metering level can be beneficial. Such a system would be valuable for both consumers and utilities, as it would allow them to take appropriate action when impurity levels increase. Currently, utilities are incorporating various water sensing sensors and alarm systems to tackle this issue. However, in order to obtain precise measurements of changes in water quality, an intelligent software solution is needed.

This solution should have the ability to train itself to recognize existing water quality standards and trigger alarms when different types of dissolved impurities are detected.

### BRIEF SUMMARY

The following summary is provided to facilitate an understanding of some of the features of the disclosed embodiments and is not intended to be a full description. A full appreciation of the various aspects of the embodiments disclosed herein can be gained by taking the specification, claims, drawings, and abstract as a whole.

It is, therefore, one aspect of the embodiments to provide for methods and systems for impurity detection using ultrasonic sensors.

It is a further aspect to provide methods and systems that can detect impurities in water using machine learning models.

It is another aspect of the embodiments to provide for the use of ultrasonic sensors and measured time-of-fight (ToF) data for use in detecting water quality.

It is another aspect of the disclosed embodiments to provide for a methods and systems for impurity detection in smart water meters.

The aforementioned aspects and other objectives can now be achieved as described herein. In an embodiment, a method for detecting water quality, can involve: classifying the quality of water using a water meter with respect to data indicative of ultrasonic time-of-flight (ToF) change behavior due to a mixed or combination of impurities in the water; and utilizing a sequential learning unit for classification of impurities in the water.

An embodiment can further involve comprising obtaining the data indicative of ultrasonic time-of-flight change behavior from a plurality of ultrasonic sensors associated with the water meter.

An embodiment may also involve obtaining the data indicative of ultrasonic time-of-flight change behavior from at least two ultrasonic sensors associated with the water meter.

An embodiment can further involve classifying with the sequential learning unit the impurities in the water as water quality parameters including at least one of: TDS (Total Dissolved Solids), ph level, chlorine residual data, turbidity information, and total organic carbon values.

An embodiment can also involve communicating data indicative of the impurities in the water classified with a machine learning algorithm to a user through a radio frequency frame.

In an embodiment can further involve: classifying with the sequential learning unit the impurities in the water as water quality parameters including at least one of: TDS (Total Dissolved Solids), ph level, chlorine residual data, turbidity information, and total organic carbon values; and communicating the water quality parameters associated with the water to a user through a radio frequency frame.

In an embodiment, the sequential learning unit can be a machine learning algorithm.

In an embodiment, the data indicative of the classification of the impurities in the water can be based on ToF, Difference in Time-of-flight (DiffToF) and/or temperature information.

In an embodiment, an apparatus for detecting water quality, can include: an ultrasonic sensor, wherein the quality of water can be classified using a water meter with respect to data indicative of ultrasonic time-of-flight (ToF) change behavior due to a mixed or combination of impurities in the water, wherein the data indicative of the ultrasonic ToF change behavior can be obtained from the ultrasonic sensor associated with the water meter; and a sequential learning unit that can classify the impurities in the water.

In an embodiment, the sequential learning unit classify the impurities in the water as water quality parameters including at least one of: TDS (Total Dissolved Solids), ph, chlorine residual, turbidity, and total organic carbon values.

In an embodiment, the data indicative of the impurities in the water classified with a machine learning algorithm can be communicated to a user through a radio frequency frame.

In an embodiment, the sequential learning unit can classify the impurities in the water as water quality parameters including at least one of: TDS (Total Dissolved Solids), ph level, chlorine residual data, turbidity information, and total organic carbon values; and the water quality parameters associated with the water can be communicated to a user through a radio frequency frame. As discussed above, the sequential learning unit can comprise a machine learning algorithm. In addition, data indicative of the classification of the impurities in the water may be based on ToF, Difference in Time-of-flight (DiffToF) and temperature information.

In an embodiment, a system for detecting water quality, can include at least one processor and a memory, the memory storing instructions to cause the at least one processor to perform: classifying the quality of water using a water meter with respect to data indicative of ultrasonic time-of-flight (ToF) change behavior due to a mixed or combination of impurities in the water; and utilizing a sequential learning unit for classification of impurities in the water.

In an embodiment, the instructions can be further configured to cause the at least one processor to perform: obtaining the data indicative of ultrasonic time-of-flight change behavior from a plurality of ultrasonic sensors associated with the water meter.

In an embodiment, the instructions can be further configured to cause the at least one processor to perform: obtaining the data indicative of ultrasonic time-of-flight change behavior from at least two ultrasonic sensors associated with the water meter.

In an embodiment, the instructions can be further configured to cause the at least one processor to perform: classifying with the sequential learning unit the impurities in the water as water quality parameters including at least one of: TDS (Total Dissolved Solids), ph level, chlorine residual data, turbidity information, and total organic carbon values; and communicating the water quality parameters associated with the water to a user through a radio frequency frame.

### BRIEF DESCRIPTION OF THE DRAWINGS

The accompanying figures, in which like reference numerals refer to identical or functionally-similar elements throughout the separate views and which are incorporated in and form a part of the specification, further illustrate the embodiments and, together with the detailed description, serve to explain the principles of the embodiments.

FIG. 1 illustrates a flow chart of operations depicting logical operational steps of a method for static meter processing, which may be implemented in accordance with an embodiment;

FIG. 2 illustrates a table indicating example data collected as part of water quality detection operations, in accordance with an embodiment;

FIG. 3 illustrates a table of water impurity visualization, in accordance with an embodiment;

FIG. 4 illustrates a schematic diagram of an impurity detection model, which may be implemented in accordance with an embodiment;

FIG. 5 illustrates a flow chart of operations depicting a method of data preprocessing, which may be implemented in accordance with an embodiment;

FIG. 6 illustrates a pictorial representation of an example of dataset preprocessing, in accordance with an embodiment;

FIG. 7 illustrates example graphs depicting data indicative of the classification of impurities based on ToF, DiffToF and temperature information, in accordance with an embodiment;

FIG. 8 illustrates a graphical representation of a model feature space, in accordance with an embodiment;

FIG. 9 illustrates graphs depicting data indicative of (Smart Water Impurity Classifier) SWIC training loss and accuracy, in accordance with an embodiment;

FIG. 10 illustrates an example of a confusion matrix, which may be implemented in accordance with an embodiment;

FIG. 11 illustrates a schematic diagram of a hybrid installation for a commercial and domestic water supply, in accordance with an embodiment;

FIG. 12 illustrates a schematic diagram of a commercial installation over a loop line for stable water flow, which can be also used as redundant supply line with metering and quality measurement capability, in accordance with an embodiment; and

FIG. 13 illustrates a schematic diagram of an example operating environment in accordance with one or more implementations described herein.

Like reference numerals or reference symbols in the various drawings may indicate like or similar elements.

### DETAILED DESCRIPTION

The particular values and configurations discussed in these non-limiting examples can be varied and are cited merely to illustrate one or more embodiments and are not intended to limit the scope thereof.

Subject matter will now be described more fully hereinafter with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other issues, subject matter may be embodied as methods, devices, components, or systems. Accordingly, embodiments may, for example, take the form of hardware, software, firmware, or a combination thereof. The following detailed description is, therefore, not intended to be interpreted in a limiting sense.

Throughout the specification and claims, terms may have nuanced meanings suggested or implied in context beyond an explicitly stated meaning. Likewise, phrases such as “in an embodiment” or “in one embodiment” or “in an example embodiment” and variations thereof as utilized herein may or may not necessarily refer to the same embodiment and the phrase “in another embodiment” or “in another example embodiment” and variations thereof as utilized herein may or may not necessarily refer to a different embodiment. It is intended, for example, that claimed subject matter include combinations of example embodiments in whole or in part.

In general, terminology may be understood, at least in part, from usage in context. For example, terms such as “and,” “or,” or “and/or” as used herein may include a variety of meanings that may depend, at least in part, upon the context in which such terms are used. Generally, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures, or characteristics in a plural sense. Similarly, terms such as “a,” “an,” or “the”, again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The term “ultrasonic sensor” as utilized herein can relate to a type of device that uses sound waves with frequencies higher than the upper audible limit of human hearing (e.g., above 20,000 hertz or 20 kHz) to detect and measure objects, distances, or other attributes. Ultrasonic sensors work on the principle of emitting high-frequency sound waves, which bounce off objects and return to the sensor. By measuring the time it takes for the sound waves to travel to the object and back, the sensor can calculate the distance to the object with high precision.

In the context of a static smart water meter, an ultrasonic sensor can be used to measure the flow rate and volume of water passing through the meter. The ultrasonic sensor can emit a burst of high-frequency sound waves (ultrasonic pulses) into the water flow. These sound waves travel through the water and bounce off the surface of the water or any particles or impurities suspended in the water. The ultrasonic sensor can measure the time it takes for the emitted sound waves to be reflected back to the sensor. This time measurement is extremely accurate and depends on the distance the sound waves traveled.

By knowing the speed of sound in water and the time it takes for the sound waves to return, the ultrasonic sensor can calculate the distance the sound traveled and, consequently, the flow rate of water passing through the meter. Over time, the ultrasonic sensor accumulates these flow rate measurements to calculate the total volume of water consumed.

Static smart water meters equipped with ultrasonic sensors offer several advantages, such as high accuracy, the ability to measure bidirectional flow (e.g., water going in and out of a property), and resistance to wear and tear since there are no moving parts. They are also suitable for various water types, including clean or contaminated water, making them a reliable choice for monitoring water consumption and quality. These meters can be integrated into smart water management systems that can provide real-time data and analytics to help utilities and consumers manage water usage more efficiently.

Note that the term “meter” as utilized herein can relate to water maters and more particularly to a “smart” water meter also known as a smart water utility meter, which is a digital device used to measure and monitor water consumption in homes, businesses, and other locations. Unlike traditional water meters that require manual reading and often provide limited data, smart water meters are equipped with advanced technology to automatically collect, record, and transmit water usage data. Key features and benefits of smart water meters include the ability to transmit water usage data remotely to a central server or utility company. This eliminates the need for physical visits to the location for meter reading. In addition, users, utility companies, and property owners can access real-time data on water consumption, allowing for better management and conservation of water resources.

Smart meters can also be used to detect abnormal usage patterns, indicating potential leaks or water wastage. This early detection can help reduce water bills and prevent property damage. Smart meters are generally more accurate than traditional mechanical meters, leading to more precise billing and data collection. Utility companies can streamline their billing processes by automatically receiving consumption data from smart meters, reducing errors and disputes.

In addition, with access to real-time usage data, consumers can become more aware of their water consumption habits, potentially leading to reduced water wastage and conservation efforts. Smart water meters can be part of a broader IoT (Internet of Things) ecosystem, enabling automated control of water-related systems, such as irrigation or water heating, based on real-time data. The collected data can be used for data analytics to identify trends, make predictions, and optimize water distribution systems. The disclosed approach described herein permits smart meters to also monitor water quality and detect contaminants, providing additional safety and environmental benefits.

As will be discussed in more detail herein, a low-power static water meter can perform a metrology measurement without having any movable parts installed. Ultrasonic sensing can be implemented with a static water meter for metrology measurements involving computation of the time-of-flight (ToF) of ultrasonic waves between a transmitter and a receiver and the calculation of flow rate information.

Predominantly ToF variations may occur due to water flow wherein the temperature and the water quality also play a significant role. This additional information can be used as part of the water quality determination process. The water temperature can be computed by existing electronic water meters, whereas water quality is supplemental information that needs extra processing for accurate detection. The disclosed water quality detection approach can thus utilize ToF information, which is generated by ultrasonic sensing, and can be subject to software classification to detect different types of impurities/quality details.

The disclosed embodiments can provide additional capabilities for impurity detection in static smart water meters. This capability can be based on ultrasonic sensing involving time-of-flight (ToF) information and computation of the water flow rate. The quality of water is an additional parameter which can be detected using sensor output and the application of a machine learning algorithm (e.g., Support Vector Machine/Neural Network). This approach can be used to detect water contaminants such as, for example, higher TDS (Total Dissolved Solids), pH, chorine residual, turbidity, and the total organic carbon values from flowing water.

A basic principle of the disclosed solution involves learning ToF data behavior during water flow when different impurities exist and later using trained models for impurity classification during normal water flow. Data collected from an ultrasonic sensor can be provided as time-series data, which can include ToF variations as a behavioral variation due to different impurities present in water. The embodiments also include impurity removal capability at the water meter level with an additional mechanism for adequate water chlorination based on the impurity classification (e.g., if germs/parasites are detected).

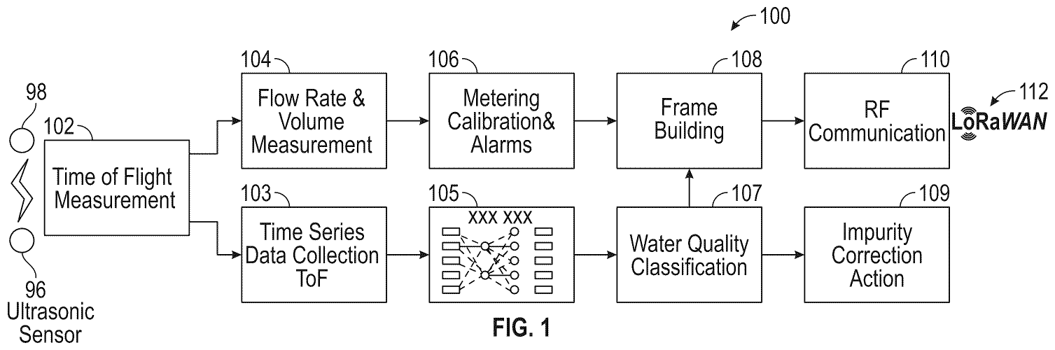

FIG. 1 illustrates a flow chart of operations depicting logical operational steps of a method 100 for static meter processing, which may be implemented in accordance with an embodiment. The method 100 shown in FIG. 1 can be implemented for impurity detection and may include a collection of time-series input data and a sequential learning unit for training and predicting water qualities.

As shown at block 102 in FIG. 1, a time-of-flight (ToF) calculation can be implemented with respect to two or more ultrasonic sensors including a first ultrasonic sensor 96 and a second ultrasonic sensor 98. Data resulting from implementation of the step or operation shown at block 102 can be subject to a step or operation involving flow rate and volume measurement as depicted at block 104 and can also be subject to time-series data collection ToF as shown at block 103. Data resulting from the operation shown at block 104 can be subject to a step or operation involving metering calibrations and alarms, as shown at block 106. Then, as shown at block 108, a step or operation involving frame building can be implemented. That is, the operation shown at block 108 can involve constructing or building a radio frequency (RF) frame.

Note that following processing of the operation shown at block 103, the collected data resulting from this step or operation can be subject to a sequential learning unit as indicated at block 105. That is, block 105 represents a sequential learning unit, which may include or implement a deep neural network. Data output from the sequential learning unit/deep neural network indicated at block 105 can be subject to water quality classification, resulting ins water quality classification data, as indicated at block 107. This water quality classification data can be provided to the frame building operation shown at block 108 and can also be subject to an impurity correction action, as shown at block 109. Data resulting from the frame building operation 108 can then be transmitted via radio frequency (RF) communication as shown at block 110 through a wireless network 11 (e.g., indicated as LoRaWAN in the figure).

In the example embodiment shown in FIG. 1, the wireless network 112 may be implemented in the contest of RF frames used in communication protocols such as, for example, LoRa (Long Range) and wMBus (Wireless M-Bus), which are structured data units used for the transmission of information over radio frequencies. These protocols can be designed for low-power, long-range wireless communication, and the frames are essential components of their communication schemes. LoRa is an example of a wireless communication technology known for its long-range capabilities and low power consumption.

Note that the embodiment shown in FIG. 1 is not limited to the specific “LoRaWAN” wireless network. “LoRaWAN” is just an example of one type of wireless based network or wireless protocol, which may be implemented with an embodiment. Embodiments may be implemented with other types of wireless networks and/or wireless protocols.

Time-of-flight (ToF) and Difference in Time-of-flight (DiffToF) timeseries data can thus be collected along with different water impurities such as hard water, RO water, salty water, acidic water, water with added protein, water with added oil components, etc. The ToF difference and variation in timeseries sequence provides classification of impurities (e.g., block 107), wherein the DiffToF can result in an accurate prediction in static water as well as in flowing water. The higher magnitude of DiffToF is an indication of flowing water. The ultrasonic sensors 96 and 98 are capable of capturing data at every second causing a high dimensional log file to be classified for detecting impurity types. Note that term “timeseries” as utilized herein can relate to “timeseries” or “time series” data, which can be a sequence of data points collected, recorded, or measured at successive points in time, typically at equally spaced intervals. Each data point in a timeseries may be associated with a specific timestamp or time period, allowing for the representation and analysis of how a particular quantity, phenomenon, or variable changes over time.

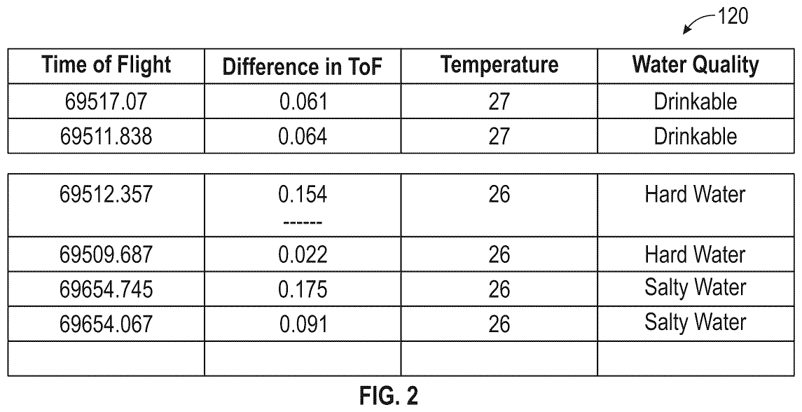

FIG. 2 illustrates a table 120 indicating example data collected as part of water quality detection operations, in accordance with an embodiment. As shown in FIG. 2, example ToF and DiffToF are respectively tracked in the first two columns, while temperature data is shown in the third column and water quality data is shown in the fourth column. Water quality data includes indications of, for example, drinkable water, hard water, salty water, and so on.

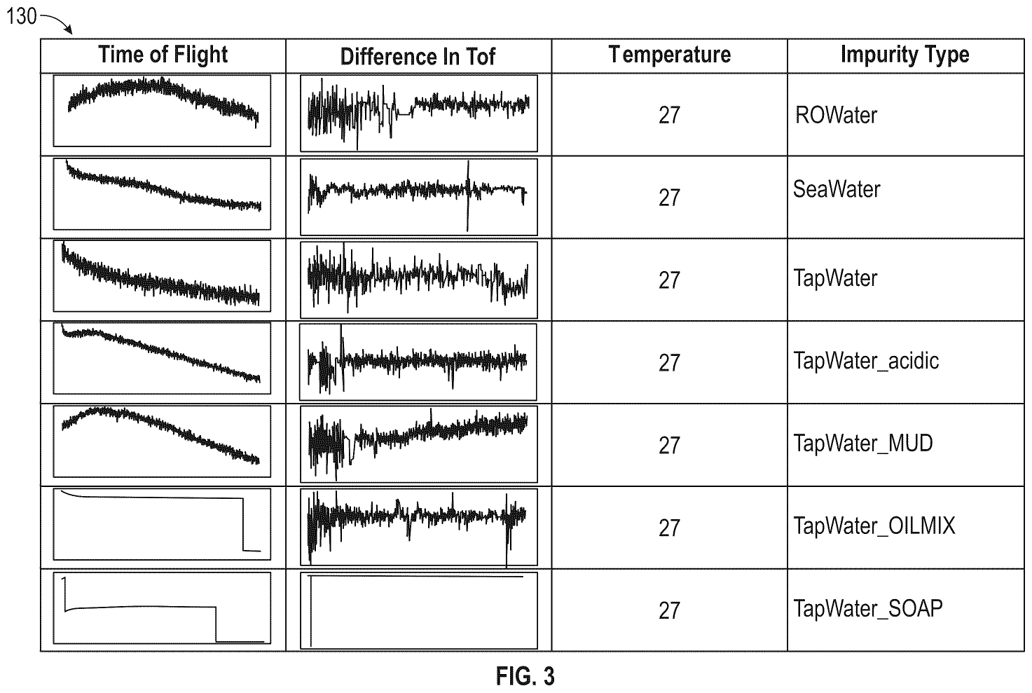

FIG. 3 illustrates a table 130 of water impurity visualization, in accordance with an embodiment. The table 130 shown in FIG. 3 includes four columns, the first of which graphically depicts different ToF with respect to graphically displayed DiffToF data in the second column. The third column tracks temperature while the fourth column displays impurities types (e.g., RO water, sea water, tap water—acidic, tap water—mud, tap water—oil mix, and tap water—soap). Table 130 thus provides a glimpse of features of individual water impurities. The example dataset shown in FIG. 3 was collected during a one hour period with data captured for each impurity at every second.

It should be appreciated that although ToF can provide some interpretable pattern of each impurity, whereas DiffToF is difficult to interpret and almost similar as data collection has been performed for static water. It is humanly impossible to interpret such a high dimensional log file continuously to monitor water condition at different geographical locations. To automatically classify timeseries data, various classifications schemes may be employed. However, as the sensor log dimensions grow exponentially, a deep neural network (DNN) appears to offer a suitable approach to meet the needs of accurate water quality detection for use as or with a sequential learning unit and/or sequential network. Unlike physical models where different numerical formulations may need to be developed and which may not be easily tuned to handle anomalous outliers, a DNN offers a data driven approaches. A DNN model can learn from complex training data the generic pattern of individual class samples by learning a suitable decision boundary or hyperplane.

The sequential learning unit shown in block 105 of FIG. 1 may in some embodiments be implemented with a DNN or other types of sequential learning units or sequential networks. Among several example DNN model types, which may be utilized to implement the sequential learning unit shown at block 105 of FIG. 1 and elsewhere herein, sequential networks may be most suitable for timeseries classification. Sequential learning units or sequential such as, for example, Recurrent Neural Network (RNN), Long-short Term Memory (LSTM), Gated Recurrent Unit (GRU) may also be used for developing a sequential network and/or a sequential learning unit as part of a sequential network.

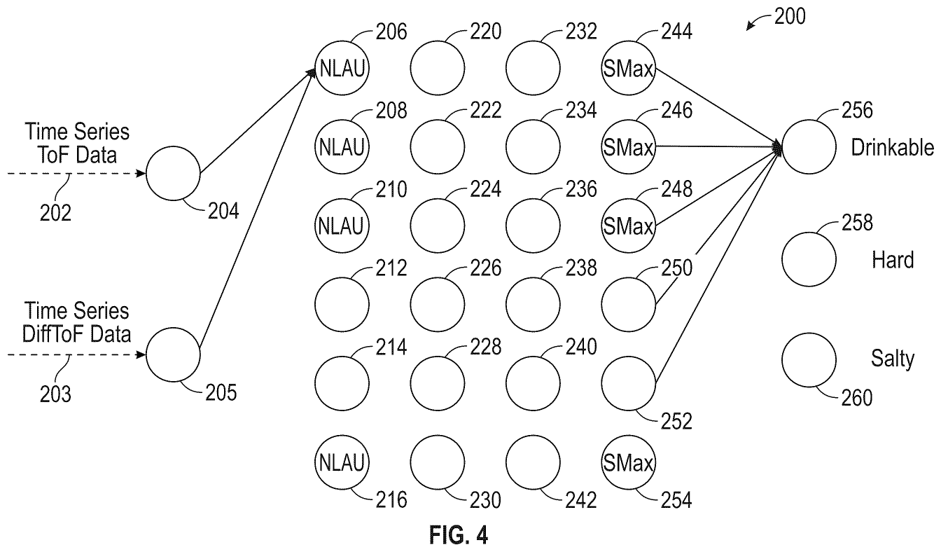

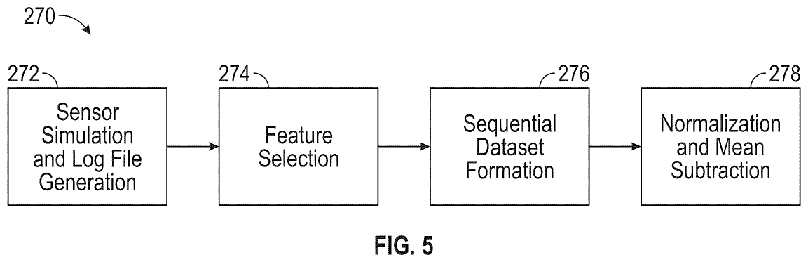

FIG. 4 illustrates a schematic diagram of an impurity detection model 200, which may be implemented in accordance with an embodiment. The impurity detection model 200 may be used to implement the sequential learning unit shown at block 105 of FIG. 1. The impurity detection model 200 can obtain ToF data as shown at dashed arrow 203 along with Timeseries DiffToF data as shown at dashed arrow 203. In the example shown in FIG. 5, the ToF data can be supplied to node 204 and the timeseries DiffToF data can be provided to node 205. Output from nodes 204 and 205 can be input to, for example, node 206 (e.g., which may be a non-linear activation unit (NLAU)). Note that nodes 206, 208, 210, and 216 are examples of non-linear activation units (NLAUs).

The impurity detection model 200 can include a number of other nodes 208 through 244. Note that in the interest of brevity, not every reference numeral and node will be discussed herein. Needless to say, data can be output from nodes 244, 246, 248, 250, 252, and 254 and input to a node 256 indicative of drinkable water (e.g., a prediction that the water is drinkable) as opposed to node 258 (e.g., hard water) and node 260 (e.g., salty water). Note that nodes 244, 246, 248, and 254 are examples of Smax nodes.

FIG. 5 illustrates a flow chart of operations depicting a method 270 of data preprocessing, which may be implemented in accordance with an embodiment. As shown at block 272, a step or operation can be implemented involving sensor simulation and log file generation. Data resulting from this operation can be subject to a feature selection step or operation as indicated at block 274. Data resulting from the feature selection operation shown at block 274 can be subject to a sequential dataset formation operation, as shown at block 276. Data resulting from the sequential dataset formation operation can be then subject to a normalizing and mean subtraction step or operation as indicated at block 278.

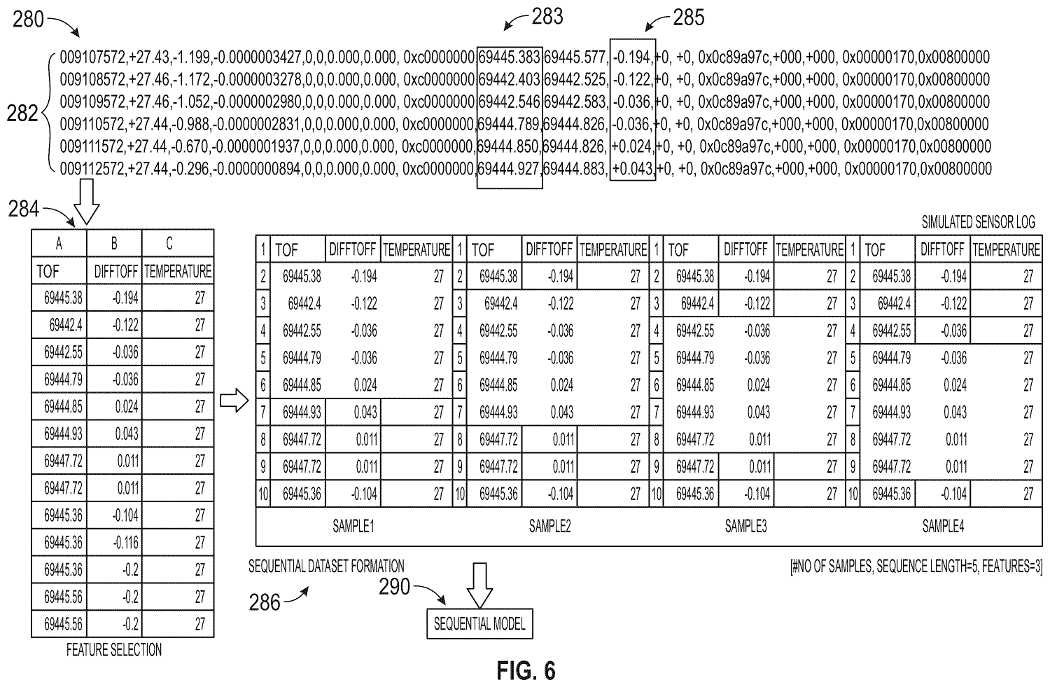

FIG. 6 illustrates a pictorial representation of an example of dataset preprocessing 280, in accordance with an embodiment. A simulated sensor log 282 is shown at the top of the figure. Example log data is shown at sections 283 and 285. Feature selection data 284 is also shown in FIG. 6, along with sequential dataset formation 286, which is then fed to a sequential model 290. The sequential model 290 is an example of a sequential learning unit such as the sequential learning unit shown at block 105 in FIG. 1.

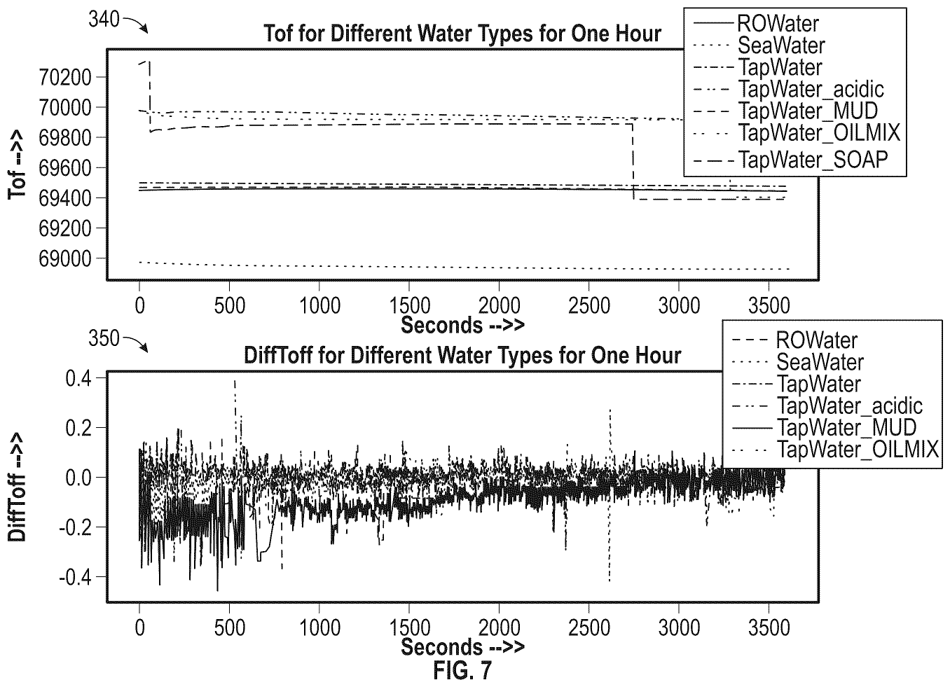

FIG. 7 illustrates example graphs 340, 350 depicting data indicative of the classification of impurities based on ToF, DiffToF and temperature information, in accordance with an embodiment. In FIG. 7, the graph 340 depicts ToF data for different water types over a one hour period. The graph 350, on the other hand, depicts DiffToF data for different water types also over a one hour period. The SWIC model 300 aims to classify impurities based on ToF, DiffToF and Temperate. The univariate features are shown in graphs 340, 350.

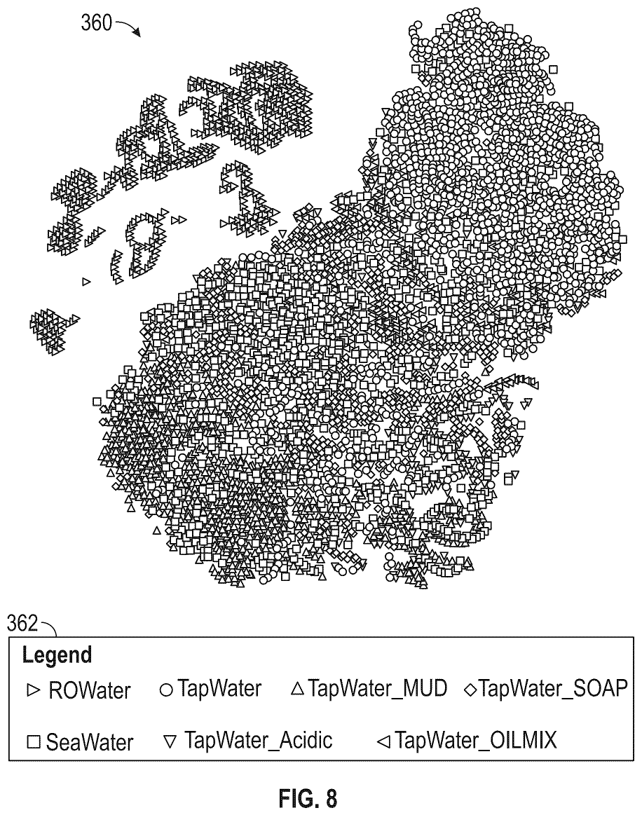

FIG. 8 illustrates a graphical representation of a model feature space 360, in accordance with an embodiment. The temporal dependency of individual classes learnt by SWIC model 300 and in model feature space can be viewed in some embodiments with samples of the same class are shown in same color. It should be noted, however, that FIG. 8 is depicted in black and white and thus lacks a particular color classification scheme. Instead, in the example shown in FIG. 8, different classes or different types of data are denoted by a geometric feature representation of different geometric icons such as squares, triangles, circles, and so on.

FIG. 8 presents an idealized and illustrative view of a model feature space and accompany example legend. Legend 362 depicted in FIG. 8 indicates a variety of different data labels such as ‘ROWater’, ‘SeaWater’, ‘TapWater’, ‘TapWater_acidic’, ‘TapWater_MUD’, ‘TapWater_OILMIX’, and ‘TapWater_SOAP’ to name a few examples, which may be associated with the model feature space 360. It should be appreciated that the particular legend 362 with its example geometric feature representations in association with the model feature space 360 as shown in FIG. 8 are provided for exemplary and illustrative purposes only and should not be considered limiting features of the embodiments.

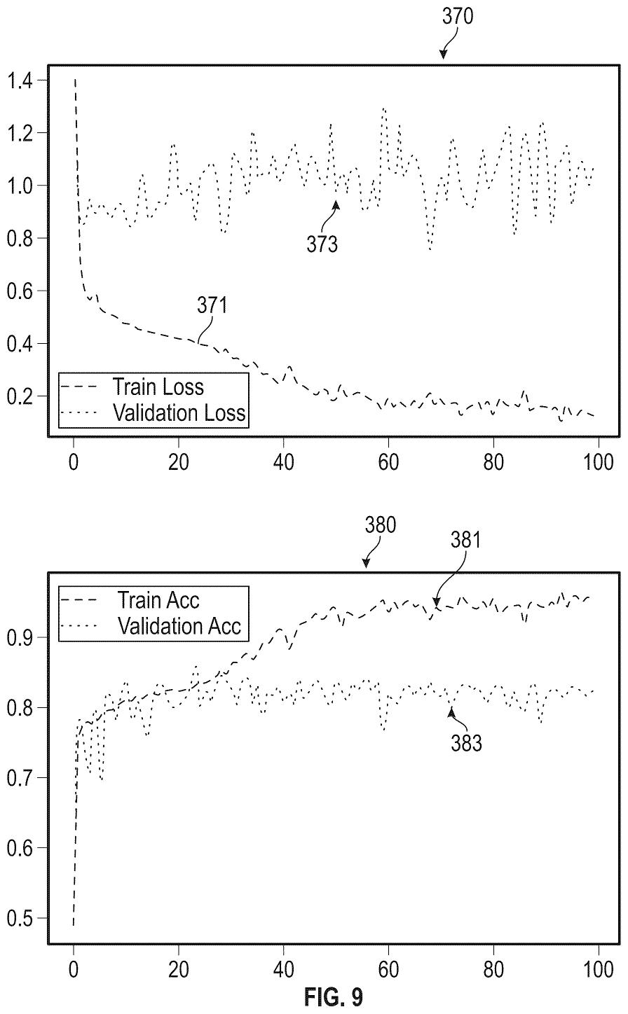

FIG. 9 illustrates graphs 370 and 380, which depict data indicative of SWIC training loss and accuracy, in accordance with an embodiment. The data shown in these graphs are based on an SWIC trained with 50 minutes of data for 7 classes and validated 10 minutes of data wherein each sample is composed of 30-time steps in temporal dimension. The SWIC training loss and accuracy is thus shown in graphs 370 and 380 in FIG. 9. For example, in graph 370 training loss is indicated by data plot 371 and validation loss is indicated by data plot 373. In graph 380, training accuracy is indicated by data plot 381 and validation accuracy is indicated by data plot 383.

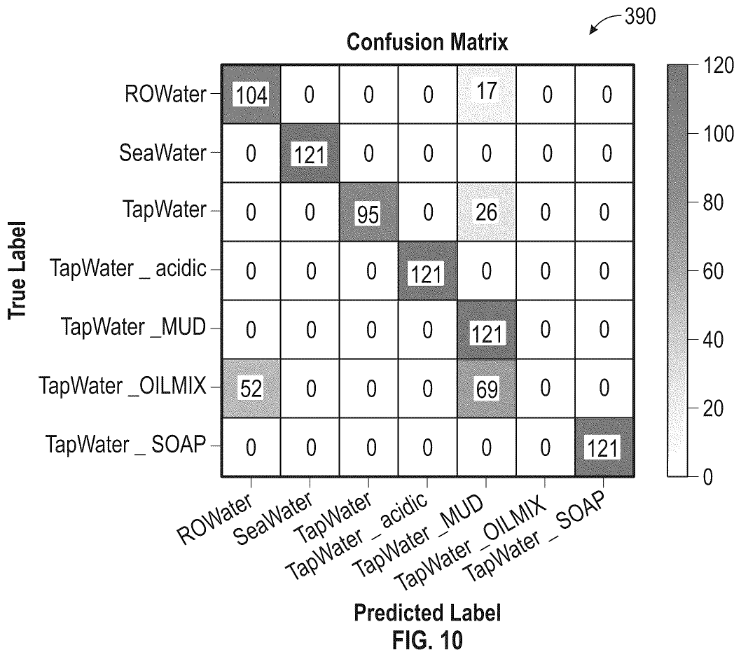

FIG. 10 illustrates an example of a confusion matrix 390, which may be implemented in accordance with an embodiment. That is, a test was performed for 2.5 minutes of data having each test sample of 30 seconds data. The SWIC model 300 is an example of a sequential learning unit such as the sequential learning unit 105 shown in FIG. 1, which can be used to classify water impurity data with, for example, an 81% classification accuracy. The confusion matrix 390 for True and Predicted classes is shown in FIG. 10. The term “confusion matrix” as utilized herein can relate to a table used in machine learning and statistics to evaluate the performance of a classification algorithm. The confusion matrix may be particularly useful for assessing the accuracy of a model's predictions when dealing with binary or multiclass classification problems. The confusion matrix can provide a comprehensive summary of how well a model has classified instances from the dataset by breaking down the results into categories.

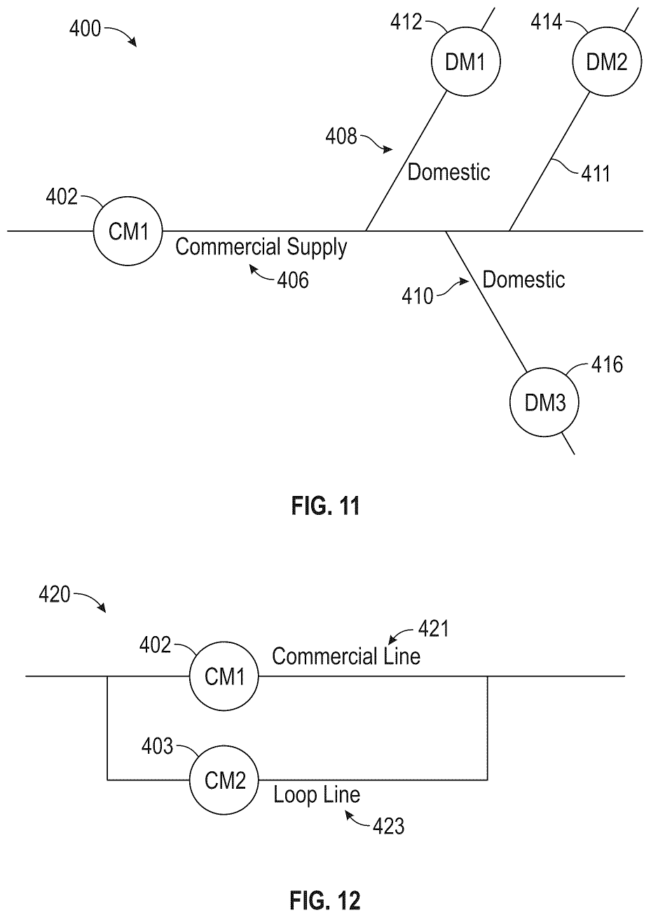

FIG. 11 illustrates a schematic diagram of a hybrid installation 400 for a commercial water supply and a domestic water supply, in accordance with an embodiment. In the configuration shown in FIG. 11, a commercial water meter 402 is shown as connected to a commercial water supply line 406, which branches into domestic water supply line 408 that connects to a domestic water meter 412. An additional domestic water supply line 410 is also shown as connected to a domestic water mater 416. A domestic water supply line 410 further connects to a domestic water meter 414. It should be appreciated that one or more ultrasonic sensors as discussed earlier can be integrated with each water meter and/or may be associated with each water meter. It should further be appreciated that the various water meters discussed above (and similarly below) may be implemented as static water meters in some embodiments.

FIG. 12 illustrates a schematic diagram of a commercial installation 420 over a loop line 423 for stable water flow, which can be also used as redundant supply line with metering and quality measurement capability, in accordance with an embodiment. The commercial installation 420 shown in FIG. 14 includes a commercial water line 421 connected to a commercial water meter 402 and the loop line 423 connected to the commercial water meter 403.

The embodiments described herein can thus provide additional capabilities for impurity detection in static water meters and can be based on the use of ultrasonic sensing technologies. The quality of water is also a possible parameter, which can be detected using sensor output with the application of additional machine learning algorithms and the detection of water contaminations such as higher TDS (Total Dissolved Solids), pH, chorine residual, turbidity, and total organic carbon values from flowing water.

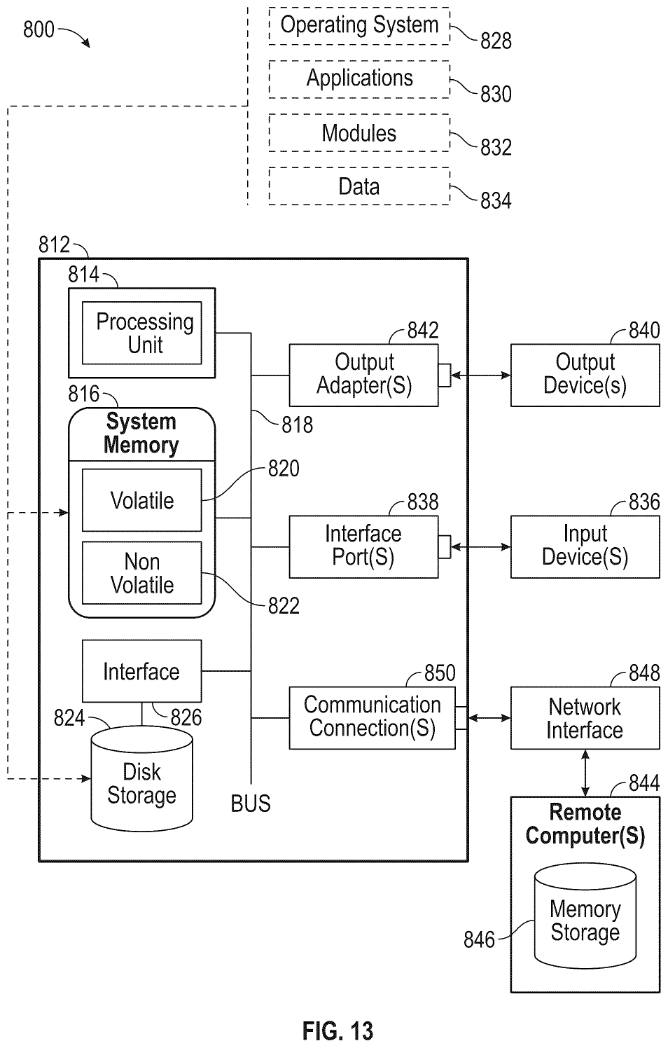

A basic principle of the embodiments involves learning ToF data behavior during water flow when different impurities exist and later using trained models for impurity classification during normal water flow. Data collected from the ultrasonic sensor(s) is timeseries data which can include a ToF variation as a behavioral variation due to different impurities present in water. The embodiments can be used for domestic as well as commercial water supply purposes and can be installed in a variety of different arrangements such as the hybrid installation solution shown in FIG. 13 and the commercial installation over a loop line for stable water flow as shown in FIG. 14 (which can be also used as redundant supply line with metering and quality measurement).

FIG. 13 illustrates an example of a suitable operating environment 800 for implementing various aspects of this disclosure and which can also include a computer 812. The computer 812 can also include a processing unit 814, a system memory 816, and a system bus 818. The system bus 818 couples system components including, but not limited to, the system memory 816 to the processing unit 814. The processing unit 814 can be any of various available processors. Dual microprocessors and other multiprocessor architectures also can be employed as the processing unit 814.

The system bus 818 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Firewire (IEEE 1094), and Small Computer Systems Interface (SCSI). The system memory 816 can also include volatile memory 820 and nonvolatile memory 822. The basic input/output system (BIOS), containing the basic routines to transfer information between elements within the computer 812, such as during start-up, is stored in nonvolatile memory 822.

By way of illustration, and not limitation, nonvolatile memory 822 can include read only memory (ROM), programmable ROM (PROM), electrically programmable ROM (EPROM), electrically erasable programmable ROM (EEPROM), flash memory, or nonvolatile random-access memory (RAM) (e.g., ferroelectric RAM (FeRAM). Volatile memory 820 can also include random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM.

Computer 812 can also include removable/non-removable, volatile/non-volatile computer storage media. FIG. 13 illustrates, for example, a disk storage 824. Disk storage 824 can also include, but is not limited to, devices like a magnetic disk drive, floppy disk drive, tape drive, Jaz drive, Zip drive, LS-100 drive, flash memory card, or memory stick. The disk storage 824 also can include storage media separately or in combination with other storage media including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 824 to the system bus 818, a removable or non-removable interface is typically used, such as interface 826.

FIG. 13 also depicts software that acts as an intermediary between users and the basic computer resources described in the suitable operating environment 800. Such software can also include, for example, an operating system 828. Operating system 828, which can be stored on disk storage 824, acts to control and allocate resources of the computer 812. System applications 830 can take advantage of the management of resources by operating system 828 through program modules 832 and program data 834, e.g., stored either in system memory 816 or on disk storage 824. It is to be appreciated that this disclosure can be implemented with various operating systems or combinations of operating systems. A user enters commands or information into the computer 812 through input device(s) 836. Input device(s) 836 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices can connect to the processing unit 814 through the system bus 818 via interface port(s) 838. Interface port(s) 838 can include, for example, a serial port, a parallel port, a game port, and a universal serial bus (USB). Output device(s) 840 may use some of the same type of ports as input device(s) 836.

Thus, for example, a USB port can be used to provide input to computer 812, and to output information from computer 812 to an output device 840. Output adapter 842 is provided herein to illustrate that there may be some output devices 840 like monitors, speakers, and printers, among other output devices 840, which require special adapters. The output adapters 842 include, by way of illustration and not limitation, video and sound cards that provide a means of connection between the output device 840 and the system bus 818. It should be noted that other devices and/or systems of devices provide both input and output capabilities such as remote computer(s) 844.

Computer 812 can operate in a networked environment using logical connections to one or more remote computers, such as remote computer(s) 844. The remote computer(s) 844 can be a computer, a server, a router, a network PC, a workstation, a microprocessor-based appliance, a peer device or other common network node and the like, and typically can also include many or all the elements described relative to computer 812. For purposes of brevity, only a memory storage device 846 is illustrated with remote computer(s) 844. Remote computer(s) 844 can be logically connected to computer 812 through a network interface 848 and then can be physically connected via communication connection 850.

Network interface 848 encompasses wire and/or wireless communication networks such as local-area networks (LAN), wide-area networks (WAN), cellular networks, etc. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL). Communication connection(s) 850 refers to the hardware/software employed to connect the network interface 848 to the system bus 818. While communication connection 850 is shown for illustrative clarity inside computer 812, it can also be external to computer 812. The hardware/software for connection to the network interface 848 can also include, for exemplary purposes only, internal and external technologies such as, modems including regular telephone grade modems, cable modems and DSL modems, ISDN adapters, and Ethernet cards.

Embodiments may be implemented in or as a system, a method, an apparatus and/or a computer program product at any possible technical detail level of integration. The computer program product can include a computer readable storage medium (or media) having computer readable program instructions thereon for causing a processor to carry out aspects of the embodiments. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing.

A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in one or more computing/processing device receives computer readable program instructions from the network and forwards the computer readable program instructions for storage in a computer readable storage medium within the respective computing/processing device.

Computer readable program instructions for carrying out operations of various aspects of the embodiments can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages.

The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to customize the electronic circuitry, to perform aspects of the embodiments.

Aspects of the embodiments are described herein with reference to flowchart illustrations and/or block diagrams of methods, apparatus (systems), and computer program products according to embodiments of the invention. It can be understood that one or more blocks of the flowchart illustrations and/or block diagrams, and combinations of blocks in the flowchart illustrations and/or block diagrams, can be implemented by computer readable program instructions. These computer readable program instructions can be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer readable program instructions can also be stored in a computer readable storage medium that can direct a computer, a programmable data processing apparatus, and/or other devices to function in a manner, such that the computer readable storage medium having instructions stored therein comprises an article of manufacture including instructions which implement aspects of the function/act specified in the flowchart and/or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the embodiments. In this regard, one or more blocks in the flowchart or block diagrams can represent a module, segment, or portion of instructions, which comprises one or more executable instructions for implementing the specified logical function(s).

In some alternative implementations of the embodiments, the functions noted in the blocks can occur out of the order noted in the figures. For example, two blocks shown in succession can, in fact, be executed substantially concurrently, or the blocks can sometimes be executed in the reverse order, depending upon the functionality involved. It can also be noted that one or more block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer and/or computers, those skilled in the art can recognize that this disclosure also can or can be implemented in combination with other program modules. Generally, program modules include routines, programs, components, data structures, etc. that perform particular tasks and/or implement abstract data types. Moreover, those skilled in the art can appreciate that the inventive computer-implemented methods can be practiced with other computer system configurations, including single-processor or multiprocessor computer systems, mini-computing devices, mainframe computers, as well as computers, hand-held computing devices (e.g., PDA, cellular phone, etc.), microprocessor-based or programmable consumer or industrial electronics, and the like. The illustrated aspects can also be practiced in distributed computing environments where tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and/or a computer.

By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal).

As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a server computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random-access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems, computer program products, and computer-implemented methods. It is, of course, not possible to describe every conceivable combination of components, products and/or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim. The descriptions of the various embodiments have been presented for purposes of illustration but are not intended to be exhaustive or limited to the embodiments disclosed. Many modifications and variations can be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Based on the foregoing, it can be appreciated that a number of different embodiments are disclosed herein including preferred and alternative embodiments. For example, in an embodiment, a method for detecting water quality, can involve: classifying the quality of water using a water meter with respect to data indicative of ultrasonic time-of-flight (ToF) change behavior due to a mixed or combination of impurities in the water; and utilizing a sequential learning unit for classification of impurities in the water.

An embodiment can further involve comprising obtaining the data indicative of ultrasonic time-of-flight change behavior from a plurality of ultrasonic sensors associated with the water meter.

An embodiment may also involve obtaining the data indicative of ultrasonic time-of-flight change behavior from at least two ultrasonic sensors associated with the water meter.

An embodiment can further involve classifying with the sequential learning unit the impurities in the water as water quality parameters including at least one of: TDS (Total Dissolved Solids), ph level, chlorine residual data, turbidity information, and total organic carbon values.

An embodiment can also involve communicating data indicative of the impurities in the water classified with a machine learning algorithm to a user through a radio frequency frame.

In an embodiment can further involve: classifying with the sequential learning unit the impurities in the water as water quality parameters including at least one of: TDS (Total Dissolved Solids), ph level, chlorine residual data, turbidity information, and total organic carbon values; and communicating the water quality parameters associated with the water to a user through a radio frequency frame.

In an embodiment, the sequential learning unit can be a machine learning algorithm.

In an embodiment, the data indicative of the classification of the impurities in the water can be based on ToF, Difference in Time-of-flight (DiffToF) and/or temperature information.

In an embodiment, an apparatus for detecting water quality, can include: an ultrasonic sensor, wherein the quality of water can be classified using a water meter with respect to data indicative of ultrasonic time-of-flight (ToF) change behavior due to a mixed or combination of impurities in the water, wherein the data indicative of the ultrasonic ToF change behavior can be obtained from the ultrasonic sensor associated with the water meter; and a sequential learning unit that can classify the impurities in the water.

In an embodiment, the sequential learning unit classify the impurities in the water as water quality parameters including at least one of: TDS (Total Dissolved Solids), ph, chlorine residual, turbidity, and total organic carbon values.

In an embodiment, the data indicative of the impurities in the water classified with a machine learning algorithm can be communicated to a user through a radio frequency frame.

In an embodiment, the sequential learning unit can classify the impurities in the water as water quality parameters including at least one of: TDS (Total Dissolved Solids), ph level, chlorine residual data, turbidity information, and total organic carbon values; and the water quality parameters associated with the water can be communicated to a user through a radio frequency frame. As discussed above, the sequential learning unit can comprise a machine learning algorithm. In addition, data indicative of the classification of the impurities in the water may be based on ToF, Difference in Time-of-flight (DiffToF) and temperature information.

In an embodiment, a system for detecting water quality, can include at least one processor and a memory, the memory storing instructions to cause the at least one processor to perform: classifying the quality of water using a water meter with respect to data indicative of ultrasonic time-of-flight (ToF) change behavior due to a mixed or combination of impurities in the water; and utilizing a sequential learning unit for classification of impurities in the water.

In an embodiment, the instructions can be further configured to cause the at least one processor to perform: obtaining the data indicative of ultrasonic time-of-flight change behavior from a plurality of ultrasonic sensors associated with the water meter.

In an embodiment, the instructions can be further configured to cause the at least one processor to perform: obtaining the data indicative of ultrasonic time-of-flight change behavior from at least two ultrasonic sensors associated with the water meter.

In an embodiment, the instructions can be further configured to cause the at least one processor to perform: classifying with the sequential learning unit the impurities in the water as water quality parameters including at least one of: TDS (Total Dissolved Solids), ph level, chlorine residual data, turbidity information, and total organic carbon values; and communicating the water quality parameters associated with the water to a user through a radio frequency frame.

It will be appreciated that variations of the above-disclosed and other features and functions, or alternatives thereof, may be desirably combined into many other different systems or applications. It will also be appreciated that various presently unforeseen or unanticipated alternatives, modifications, variations or improvements therein may be subsequently made by those skilled in the art which are also intended to be encompassed by the following claims.

What is claimed is:

1. A method for detecting water quality, comprising:
classifying a quality of water using a water meter with respect to data indicative of ultrasonic time-of-flight (ToF) change behavior due to a mixed or combination of impurities in the water; and
utilizing a sequential learning unit for classification of impurities in the water.

2. The method of claim 1 further comprising obtaining the data indicative of ultrasonic time-of-flight change behavior from a plurality of ultrasonic sensors associated with the water meter.

3. The method of claim 1 further comprising obtaining the data indicative of ultrasonic time-of-flight change behavior from at least two ultrasonic sensors associated with the water meter.

4. The method of claim 1 further comprising:
classifying with the sequential learning unit the impurities in the water as water quality parameters including at least one of: TDS (Total Dissolved Solids), ph level, chlorine residual data, turbidity information, and total organic carbon values.

5. The method of claim 1 further comprising:
communicating data indicative of the impurities in the water classified with a machine learning algorithm to a user through a radio frequency frame.

6. The method of claim 1 further comprising
classifying with the sequential learning unit the impurities in the water as water quality parameters including at least one of: TDS (Total Dissolved Solids), ph level, chlorine residual data, turbidity information, and total organic carbon values; and
communicating the water quality parameters associated with the water to a user through a radio frequency frame.

7. The method of claim 1 wherein the sequential learning unit comprises a machine learning algorithm.

8. The method of claim 1 wherein data indicative of the classification of the impurities in the water is based on ToF, Difference in Time-of-flight (DiffToF) and temperature information.

9. An apparatus for detecting water quality, comprising:
an ultrasonic sensor, wherein a quality of water is classified using a water meter with respect to data indicative of ultrasonic time-of-flight (ToF) change behavior due to a mixed or combination of impurities in the water, wherein the data indicative of the ultrasonic ToF change behavior is obtained from the ultrasonic sensor associated with the water meter; and
a sequential learning unit that classifies the impurities in the water.

10. The apparatus of claim 10 wherein the sequential learning unit classifies the impurities in the water as water quality parameters including at least one of: TDS (Total Dissolved Solids), ph, chlorine residual, turbidity, and total organic carbon values.

11. The apparatus of claim 10 wherein data indicative of the impurities in the water classified with a machine learning algorithm is communicated to a user through a radio frequency frame.

12. The apparatus of claim 10 wherein:
the sequential learning unit classifies the impurities in the water as water quality parameters including at least one of: TDS (Total Dissolved Solids), ph level, chlorine residual data, turbidity information, and total organic carbon values; and
the water quality parameters associated with the water are communicated to a user through a radio frequency frame.

13. The apparatus of claim 10 wherein the sequential learning unit comprises a machine learning algorithm.

14. The apparatus of claim 1 wherein data indicative of the classification of the impurities in the water is based on ToF, Difference in Time-of-flight (DiffToF) and temperature information.

15. A system for detecting water quality, comprising:
at least one processor and a memory, the memory storing instructions to cause the at least one processor to perform:
classifying a quality of water using a water meter with respect to data indicative of ultrasonic time-of-flight (ToF) change behavior due to a mixed or combination of impurities in the water; and
utilizing a sequential learning unit for classification of impurities in the water.

16. The system of claim 15 wherein the instructions are further configured to cause the at least one processor to perform: obtaining the data indicative of ultrasonic time-of-flight change behavior from a plurality of ultrasonic sensors associated with the water meter.

17. The system of claim 15 wherein the instructions are further configured to cause the at least one processor to perform: obtaining the data indicative of ultrasonic time-of-flight change behavior from at least two ultrasonic sensors associated with the water meter.

18. The system of claim 15 wherein the instructions are further configured to cause the at least one processor to perform:
classifying with the sequential learning unit the impurities in the water as water quality parameters including at least one of: TDS (Total Dissolved Solids), ph level, chlorine residual data, turbidity information, and total organic carbon values; and
communicating the water quality parameters associated with the water to a user through a radio frequency frame.

19. The system of claim 15 wherein the sequential learning unit comprises a machine learning algorithm.

20. The system of claim 15 wherein data indicative of the classification of the impurities in the water is based on ToF, Difference in Time-of-flight (DiffToF) and temperature information.
